(19) United States
(12) Patent Application Publication
(10) Pub. No.: US 2024/0189257 A1
(43) Pub. Date: Jun. 13, 2024

(54) COMPOSITIONS AND METHODS FOR MANAGING NEPHROPATHY

(71) Applicant: SAMI-SABINSA GROUP LIMITED, BANGALORE (IN)

(72) Inventors: Muhammed Majeed, Bangalore (IN); Kalyanam Nagabhushanam, East Windsor, NJ (US); Sarang Bani, Bangalore (IN); Anjali Pandey, Bangalore (IN)

(21) Appl. No.: 18/282,672

(22) PCT Filed: Mar. 25, 2021
(86) PCT No.: PCT/US2021/024125

§ 371 Date: Sep. 18, 2023

(51) Int. Cl. A61K 31/12; A61K 31/37; A61K 31/7034; A61K 36/185; A61K 36/48; A61P 13/12

(52) U.S. Cl. CPC A61K 31/12; A61K 31/37; A61K 31/7034; A61K 36/185; A61K 36/48; A61P 13/12

(57) ABSTRACT

The invention discloses the use of a composition comprising 70%-80% w/w tetrahydrocurcuminoids, 10%-20% w/w hexahydrocurcuminoids and 5%-10% w/w octahydrocurcuminoids in the therapeutic management of nephropathy in mammals. The composition further comprises β-glucogallin and total mucic acid gallates, C-glucosides and tannins isolated from Pterocarpus marsupium. The invention also discloses the potential of the composition comprising β-glucogallin and total mucic acid gallates from Emblica officinalis, C-glucosides and tannins isolated from Pterocarpus marsupium in the therapeutic management of nephropathy.

### CROSS-REFERENCE TO RELATED APPLICATIONS

This is a US national phase application of PCT application no. PCT/US2021/024125, filed on 25 Mar. 2021, the contents of which is incorporated herein in its entirety.

### FIELD OF INVENTION

The present invention relates to a compositions and methods for managing nephropathy. More specifically the invention relates to the potential of a composition comprising curcuminoids composition standardised to contain tetrahydrocurcuminoids (THCs), hexahydrocurcuminoids (HHCs) and octahydrocurcuminoids (OHCs), for the therapeutic management of nephropathy. The composition further comprises β-glucogallin and total mucic acid gallates, C-glucosides and tannins isolated from Pterocarpus marsupium.

### BACKGROUND OF THE INVENTION

### Description of Prior Art

Nephropathy is kidney disorder caused by damage to the small blood vessels or to the nephrons in the kidneys that are vital in cleaning blood and excreting waste products. There are many causes for the development of nephropathy which include, high blood sugar (hyperglycemia), high blood pressure, high cholesterol, a family history of diabetes and kidney diseases. Increased inflammation also leads to the loss in the kidney function and further lead to the development of nephropathy. Nephropathy is also caused by certain contrast agents and media (Golshahi et al., Contrast-induced nephropathy; A literature review, J Nephropathol. 2014; 3(2): 51-56). Nephropathy results in improper waste removal by the kidneys, renal insufficiency, and presence of protein in urine which is an indicator of renal failure. The risk of nephropathy also increases with age where there is a gradual loss of kidney function.

The following prior art documents discloses the various pathological aspects of nephropathy.
a. Ayodele et al., Diabetic nephropathy—a review of the natural history, burden, risk factors and treatment, J Natl Med Assoc. 2004 November; 96(11): 1445-1454
b. Papadopoulou-Marketou et al., Diabetic nephropathy in type 1 diabetes: a review of early natural history, pathogenesis, and diagnosis, Diabetes Metab Res Rev. 2017; 33:e2841.
c. Navarro-Gonzalez et al., Inflammatory molecules and pathways in the pathogenesis of diabetic nephropathy, Nat Rev Nephrol 7, 327-340 (2011). https://doi.org/10.1038/nrneph.2011.51
d. Ibels L S, Gyory A Z. IgA nephropathy: analysis of the natural history, important factors in the progression of renal disease, and a review of the literature. Medicine. 1994 March; 73(2):79-102.
e. Maeder et al., Contrast nephropathy. Review focusing on prevention, J Am Coll Cardiol. 2004 November, 44 (9) 1763-1771

The current treatment modalities include drugs for treating high blood pressure, high blood glucose, high cholesterol and drugs to reduce protein in urine. Since nephropathy is a multi-factorial disorder, a single drug which treats only one of the above conditions may not be a viable option to improve the symptoms of nephropathy. There still exists an unmet industrial need to find a composition that is effective in managing all the symptoms of nephropathy. The present invention solves this unmet need by disclosing a curcuminoid composition standardised to contain tetrahydrocurcuminoids, hexahydrocurcuminoids, and octahydrocurcuminoids. This composition is useful in addressing all the causative factors that lead to the development of nephropathy and is useful for maintaining a healthy kidney function.

It is well known in the scientific art regarding the compounds obtained from Curcuma species, specifically curcuminoids, and their role in therapeutic management of various diseases and disorders are well documented. The metabolites of curcumin—tetrahydrocurcuminoids, hexahydrocurcuminoids, and octahydrocurcuminoids are garnering much attention owing to their similar and superior efficacy over curcumin (Majeed et al., Reductive Metabolites of Curcuminoids, Nutriscience Publishers LLC, 2019). The pharmacological activities of these reductive metabolites of curcumin are yet to be proven and tapped for industrial application, specifically for managing nephropathy and other kidney disorders. The composition along with β-glucogallin and total mucic acid gallates from Emblica officinalis and C-glucosides and tannins isolated from Pterocarpus marsupium shows synergistic benefits in reducing the symptoms of nephropathy and is useful for managing normal kidney function.

It is the principle object of the invention to disclose a method for the therapeutic management of nephropathy in mammals using a composition comprising curcuminoids composition standardised to contain tetrahydrocurcuminoids, hexahydrocurcuminoids and octahydrocurcuminoids. The composition further comprises β-glucogallin and total mucic acid gallates isolated from Emblica officinalis and C-glucosides and tannins isolated from Pterocarpus marsupium.

It is another object of the invention to disclose a method for maintaining normal kidney function in mammals using a composition comprising curcuminoids composition standardised to contain tetrahydrocurcuminoids, hexahydrocurcuminoids and octahydrocurcuminoids. The composition further comprises β-glucogallin and total mucic acid gallates isolated from Emblica officinalis and C-glucosides and tannins isolated from Pterocarpus marsupium.

It is yet another object of the invention to disclose a method for the therapeutic management of nephropathy in mammals using a composition comprising β-glucogallin and total mucic acid gallates isolated from Emblica officinalis and C-glucosides and tannins isolated from Pterocarpus marsupium.

The present invention fulfils the abovementioned objects and provides further related advantages.

### SUMMARY OF THE INVENTION

In a most preferred embodiment, the invention discloses a method for the therapeutic management of nephropathy in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such therapeutic management. The composition further comprises β-glucogallin and total mucic acid gallates, C-glucosides and tannins isolated from Pterocarpus marsupium.

In another most preferred embodiment, the invention discloses a method for the maintaining normal kidney function in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such therapeutic management.

In a most preferred embodiment, the invention discloses a method for the therapeutic management of nephropathy in mammals, said method comprising step of administering a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside, to mammals in need of such therapeutic management.

Other features and advantages of the present invention will become apparent from the following more detailed description, which illustrate, by way of example, the principle of the invention.

### BRIEF DESCRIPTION OF THE DRAWINGS

FIG. 1 is a graphical representation showing the reduction in cystatin C levels in mice treated with different compositions. Values are expressed as Mean (ng/ml)

FIG. 2 is a graphical representation showing the reduction in kidney injury molecule-1 levels in mice treated with different compositions. Values are expressed as Mean (ng/ml)

FIG. 3 is a graphical representation showing the reduction in angiotensin-II levels in mice treated with different compositions. Values are expressed as Mean (pg/ml)

FIG. 4 is a graphical representation showing the reduction in NLRP-3 levels in mice treated with different compositions. Values are expressed as Mean (ng/ml)

FIG. 5 is a graphical representation showing the reduction in TGF-β levels in mice treated with different compositions. Values are expressed as Mean (ng/ml)

FIG. 6 is a graphical representation showing the reduction in MCP-1 levels in mice treated with different compositions. Values are expressed as Mean (pg/ml)

FIG. 7 is the histopathological staining image of mice kidney section showing the effectiveness of the compositions. A—Normal control, B—diabetic control, C—β glucogallin+pTerosol, D-β glucogallin+pTerosol+C3 Reduct Special

### DETAILED DESCRIPTION OF PREFERRED EMBODIMENTS

In a most preferred embodiment, the invention discloses a method for the therapeutic management of nephropathy in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such therapeutic management. In a related aspect, the tetrahydrocurcuminoids comprise of tetrahydrocurcumin, tetrahydro-demethoxycurcumin and tetrahydrobis-demethoxycurcumin. In another related aspect, the hexahydrocurcuminoids further comprise of hexahydrocurcumin, hexahydro-demethoxycurcumin and hexahydrobis-demethoxycurcumin. In yet another preferred embodiment, octahydrocurcuminoids further comprise of octahydrocurcumin, octahydro-demethoxycurcumin and octahydrobis-demethoxycurcumin. In yet another related aspect, the composition further comprises at least 10% w/w 1-O-galloyl-β-D-glucose (β-glucogallin) and at least 10% w/w total mucic acid gallates and ellagic acid, isolated from Emblica officinalis. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In further related aspect, the composition further comprises of Pterocarpus marsupium extract standardized to contain tannins and C-glycosides containing not less than 0.5% w/w Pterocarposide, not less than 0.5% w/w Sabioside. In another related aspect, nephropathy is induced by conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In another preferred embodiment, the invention discloses the use of a composition comprising 70%-80% w/w tetrahydrocurcuminoids, 10%-20% w/w hexahydrocurcuminoids and 5%-10% w/w octahydrocurcuminoids in the therapeutic management of nephropathy in mammals. In a related aspect, the tetrahydrocurcuminoids comprise of tetrahydrocurcumin, tetrahydro-demethoxycurcumin and tetrahydrobis-demethoxycurcumin. In another related aspect, the hexahydrocurcuminoids further comprise of hexahydrocurcumin, hexahydro-demethoxycurcumin and hexahydrobis-demethoxycurcumin. In yet another preferred embodiment, octahydrocurcuminoids further comprise of octahydrocurcumin, octahydro-demethoxycurcumin and octahydrobis-demethoxycurcumin. In yet another related aspect, the composition further comprises at least 10% w/w 1-O-galloyl-β-D-glucose (β-glucogallin) and at least 10% w/w total mucic acid gallates and ellagic acid, isolated from Emblica officinalis. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In further related aspect, the composition further comprises of Pterocarpus marsupium extract standardized to contain tannins and C-glycosides containing not less than 0.5% w/w Pterocarposide, not less than 0.5% w/w Sabioside. In another related aspect, nephropathy is induced by conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In another most preferred embodiment, the invention discloses a method for the maintaining normal kidney function in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such therapeutic management. In a related aspect, the tetrahydrocurcuminoids comprise of tetrahydrocurcumin, tetrahydro-demethoxycurcumin and tetrahydrobis-demethoxycurcumin.

In another related aspect, the hexahydrocurcuminoids further comprise of hexahydrocurcumin, hexahydro-demethoxycurcumin and hexahydrobis-demethoxycurcumin. In yet another preferred embodiment, octahydrocurcuminoids further comprise of octahydrocurcumin, octahydro-demethoxycurcumin and octahydrobis-demethoxycurcumin. In yet another related aspect, the composition further comprises at least 10% w/w 1-O-galloyl-β-D-glucose (β-glucogallin) and at least 10% w/w total mucic acid gallates and ellagic acid, isolated from Emblica officinalis. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In further related aspect, the composition further comprises of Pterocarpus marsupium extract standardized to contain tannins and C-glycosides containing not less than 0.5% w/w Pterocarposide, not less than 0.5% w/w Sabioside. In another related aspect, the composition maintains normal kidney function in conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In another preferred embodiment, the invention discloses the use of a composition comprising 70%-80% w/w tetrahydrocurcuminoids, 10%-20% w/w hexahydrocurcuminoids and 5%-10% w/w octahydrocurcuminoids in maintaining normal kidney function in mammals. In a related aspect, the tetrahydrocurcuminoids comprise of tetrahydrocurcumin, tetrahydro-demethoxycurcumin and tetrahydrobis-demethoxycurcumin. In another related aspect, the hexahydrocurcuminoids further comprise of hexahydrocurcumin, hexahydro-demethoxycurcumin and hexahydrobis-demethoxycurcumin. In yet another preferred embodiment, octahydrocurcuminoids further comprise of octahydrocurcumin, octahydro-demethoxycurcumin and octahydrobis-demethoxycurcumin. In yet another related aspect, the composition further comprises at least 10% w/w 1-O-galloyl-β-D-glucose (β-glucogallin) and at least 10% w/w total mucic acid gallates and ellagic acid, isolated from Emblica officinalis. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In further related aspect, the composition further comprises of Pterocarpus marsupium extract standardized to contain tannins and C-glycosides containing not less than 0.5% w/w Pterocarposide, not less than 0.5% w/w Sabioside. In another related aspect, the composition maintains normal kidney function in conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In a most preferred embodiment, the invention discloses a method for the therapeutic management of nephropathy in mammals, said method comprising step of administering a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside, to mammals in need of such therapeutic management. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In another related aspect, nephropathy is induced by conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In another preferred embodiment, the invention discloses the use of a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside in the therapeutic management of nephropathy in mammals. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In another related aspect, nephropathy is induced by conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In a most preferred embodiment, the invention discloses a method for the maintaining normal kidney function in mammals in mammals, said method comprising step of administering a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside, to mammals in need of such therapeutic management. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In another related aspect, nephropathy is induced by conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brought about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In another preferred embodiment, the invention discloses the use of a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside for maintaining normal kidney function in mammals. In another related aspect, the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate. In another related aspect, the composition maintains normal kidney functions in conditions selected from the group consisting of hyperglycemia, hypercholesterolemia, elevated blood pressure and renal hypertension, drugs, autoimmune diseases, oxidative stress and inflammation. In yet another related aspect, the therapeutic effect is brough about by improving renal insufficiency, decreasing renal hypertension, reducing diabetic complications, decreasing inflammation and reducing the circulating levels of kidney markers in blood. In a further related aspect, the composition further comprises of stabilizing agents, bioavailability enhancers and antioxidants, pharmaceutically or nutraceutically or cosmeceutically accepted excipients and enhancers. In a preferred embodiment, the mammal is human.

In yet another related embodiment the bioavailability enhancer is selected from the group consisting of, but not limited to, piperine, quercetin, garlic extract, ginger extract, and naringin.

In another related aspect, one or more anti-oxidants and anti-inflammatory agents are selected from the group consisting of, but not limited to, vitamin A, D, E, K, C, B complex, rosmarinic acid, Alpha Lipoic Acid, Ellagic Acid, Glycyrrhizinic Acid, Epigallocatechin Gallate, plant polyphenols, Glabridin, moringa oil, oleanolic acid, Oleuropein, Carnosic acid, urocanic acid, phytoene, lipoid acid, lipoamide, ferritin, desferal, billirubin, billiverdin, melanins, ubiquinone, ubiquinol, ascorbyl palmitate, Mg ascorbyl phosphate, ascorbyl acetate, tocopherols and derivatives such as vitamin E acetate, uric acid, α-glucosylrutin, calalase and the superoxide dismutase, glutathione, selenium compounds, butylated hydroxyanisole (BHA), butylated hydroxytoluene (BHT), sodium metabisulfite (SMB), propyl gallate (PG) and amino acid cysteine.

Specific illustrative examples enunciating the most preferred embodiments are included herein below.

### EXAMPLES

### Example 1: Composition

The reductive metabolites of curcuminoids viz. tetrahydrocurcuminoids, hexahydrocurcuminoids and octahydrocurcuminoids are commonly biotransformed by the reductive enzymes and by hydrogenation (Mimura et. al., U.S. Pat. No. 5,266,344; Pan et al., Biotransformation of curcumin through reduction and glucuronidation in mice, Drug Metab Dispos, 1999, 27(1):486-494). They are also identified in nature and isolated from different plant sources (Majeed et al., Reductive Metabolites of Curcuminoids, Nutriscience Publishers LLC, 2019). Tetrahydrocurcumin along with its analogues tetrahydrodemethoxycurcumin, tetrahydrobisdemethoxycurcumin has been isolated from Zingiber sp. and Curcuma sp. (Peng et al., Chemical constituents of Zingiber officinale (Zingeberaceae). Yunnan Zhiwu Yanjiu, 2007; 29(1):125-128). Tetrahydrodemethoxycurcumin and tetrahydrobisdemethoxycurcumin has been reported to be present in the rhizome of Thai Zeodary (Curcuma zedoaria) (Matsuda et al., Anti-allergic principles from Thai zedoary: structural requirements of curcuminoids for inhibition of degranulation and effect on the release of TNF-alpha and IL-4 in RBL-2H3 cells. Bioorg med hem, 2004; 12(22).5891-5898). Tetrahydrocurcumin is also obtained by biotransformation from curcumin (Shimoda et al., Formation of tetrahydrocurcumin by reduction of curcumin with cultured plant cells of Marchantia polymmrpha, Nat Prod Commun, 2012, 7(4):529-530).

Hexahydrocurcuminoids is also a naturally occurring plant metabolite found in the roots and rhizomes of Curcuma, Zingiber and Alpina. Hexahydrocurcuminoids have been isolated from rhizomes of fresh ginger (Peng et al., Cytotoxic, cytoprotective and antioxidant effects of isolated phenolic compounds from fresh ginger, Fitoterapia, 2012, 83(3):568-585). Hexahydrocurcuminoids are reported to occur in either of the two enatiomeric forms (S&R) and also as a racemic mixture (Majeed et al., Reductive Metabolites of Curcuminoids, Nutriscience Publishers LLC, 2019).

Similarly, octahydrocurcuminoids are also isolated from the rhizomes of C. xanthorrhiza (Uehara et al., Diarylheptanoids from the rhizomes of Curcuma xanthorrhiza and Alpinia officinarum, Chem Pharm Bull, 1987, 35(8):3298-3304). Octahydrocurcumin is also prepared by hydrogenation form tetrahydrocurcumin, in-vivo and microbial biotransformation (Majeed et al., Reductive Metabolites of Curcuminoids, Nutriscience Publishers LLC, 2019)

The composition claimed in the present invention was formulated according to the procedures as disclosed in US patent application no. U.S. Ser. No. 17/071,682 which is incorporated herein as reference. The composition is also available commercially as C3 Reduct® Special from Sami-Sabinsa Group Limited, Bangalore, India.

The β-glucogallin composition was isolated from Emblica officinalis as per the protocol disclosed in US 2010-0034762 A1, which is incorporated herein by reference. This is also available commercially as Saberry® from Sami-Sabinsa Group Limited, Bangalore, India. Similarly, the tannins and C-glycosides containing not less than 0.5% w/w Pterocarposide (CAS no. 264876-26-8) and not less than 0.5% w/w Sabioside (CAS no. 108351-24-2), were isolated form Pterocarpus marsupium as per the procedures described in US patent application no. U.S. Ser. No. 16/515,298, incorporated herein by reference. This is commercially available as pTerosol® from Sami-Sabinsa Group Limited, Bangalore, India

### Example 2: Therapeutic Potential of the Composition Comprising Tetrahydrocurcuminoids, Hexahydrocurcuminoids and Octahydrocurcuminoids in Managing Nephropathy

The potential of the composition was evaluated for managing nephropathy, with an illustrative example of diabetic nephropathy. A person skilled in the art will understand from below examples that the potential of the composition in managing nephropathy caused due to various reasons are considered equivalent.

### Methodology

After 1 week of adaptive feeding, the rats were randomly divided into normal control and experimental groups. The experimental group received high-fat diet (consisting of 70% standard laboratory chow, 15% carbohydrates, 10% lard, and 5% yolk powder) to establish an insulin resistance model, while the normal control group receive standard chow. After 4 weeks of high-fat diet feeding, the experimental group rats were induced with diabetes by a single intraperitoneal injection of STZ (35 mg/kg, dissolved in 0.01 M sodium citrate buffer, pH 4.4), while the Normal group rats were injected with the same dose of citrate buffer. At the 7th day after STZ injection, the blood glucose levels of rats were measured to confirm the successful establishment of the model. Rats with blood glucose level ≥250 mg/dl is indicative of the establishment of the disease and selected for further pharmacological studies. All rats were fed their respective diets until the end of the study. Rats were also given 1% NaCl (salt) to drink and on day 28, 0.75 μg aldosterone (DOCA) was subcutaneously infused. Blood pressure and urinary albumin were measured to establish renal hypertension.

All the experimental rats were assigned to the specific treatment group and test drugs were administered orally once daily between 9:00 and 11:00 a.m., continuously for 12 weeks.

The groups were administered the following:

TABLE 1
Details of composition administered

| | | | |
|---|---|---|---|
| Individual | β- Glucogallin (Saberry ®) | 50 | mg/kg p.o. |
| component | pTerosol ® | 100 | mg/kg p.o. |
| | C3 Reduct ® Special | 50 | mg/kg p.o. |
| Combination | β- Glucogallin + pTerosol ® | 150 | mg/kg p.o. |
| of two | β- Glucogallin + C3 Reduct ® Special | 100 | mg/kg p.o. |
| | pTerosol ® + C3 Reduct ® Special | 150 | mg/kg p.o. |
| Formulation | β- Glucogallin + pTerosol ® + | 200 | mg/kg p.o. |
| of three | C3 Reduct ® Special | | |
| Standard | Metformin | 100 | mg/kg p.o. |

Legend:
C3 Reduct ® Special - Curcuminoid composition containing 70%-80% w/w tetrahydrocurcuminoids, 10%-20% w/w hexahydrocurcuminoids and 5%-10% w/w octahydrocurcuminoids.
pTerosol ® - Pterocarpus marsupium extract standardized to contain tannins and C-glycosides containing not less than 0.5% w/w Pterocarposide (CAS no. 264876-26-8) and not less than 0.5% w/w Sabioside (CAS no. 108351-24-2).
Saberry ® - Composition comprising at least 10% w/w 1-O-galloyl-β-D-glucose (β-glucogallin) and at least 10% w/w total mucic acid gallates and ellagic acid, isolated from Emblica officinalis

### Urine, Blood, and Tissue Processing

At the end of the treatment, all rats were be housed in individual metabolic cages for 24 h to collect urine samples and then sacrifice. Urine samples were centrifuged at 4000×g for 5 min to separate the supernatant. Blood samples are acquired by a small puncture to retro orbital plexus and centrifuged at 4000×g for 10 min at 4° C. to separate the plasma for subsequent experiments. Serum was also separated from the blood for analysis in which anti-coagulant was not added. Thereafter, the kidneys were immediately harvested and weighed. The right kidneys were washed in ice-cold saline solution and fixed in 4% neutral formaldehyde solution for histological examination, whereas the renal cortex of the left kidneys was rinsed with cold isotonic saline and subsequently stored at −70° C. until biochemical estimations.

Analysis of blood and urine biochemical parameters viz. Blood glucose, Serum creatinine, BUN, Urine creatinine and Urinary albumin was done by Biochemical Analyzer.

Measurement of renal lipids viz. Triglyceride, Total cholesterol, Free Fatty Acid, Phospholipids was also done by automated analyzer.

Cystatin C levels were measured in serum samples collected from the experimental animals by commercially available ELISA kit, Thermo Scientific™ Pierce™ Rat Cystatin C (CST3) and the estimations were done according to the user manual. Similarly, Angiotensin II was also measured in serum sample of the animals by ELISA.

The other molecular targets, Kidney Injury Molecule-1, Transforming growth factor beta (TGF-β), Monocyte chemoattractant protein-1, Nod-like receptor protein 3 (NLRP3) inflammasome were measured in kidney tissue homogenate by commercially available ELISA kits according to the instructions provided in user's manual.

The solid-phase sandwich ELISA (enzyme-linked immunosorbent assay) is designed to measure the amount of the target bound between a matched antibody pair. A target-specific antibody has been pre-coated in the wells of the supplied microplate. Samples, standards, or controls are then added into these wells and bind to the immobilized (capture) antibody. The sandwich is formed by the addition of the second (detector) antibody, a substrate solution is added that reacts with the enzyme-antibody-target complex to produce measurable signal. The intensity of this signal is directly proportional to the concentration of target present in the original specimen.

### Kidneys Histological Examination

Kidney tissues were immediately preserved in 10% neutral buffered formalin, dehydrated through graded alcohol series, embedded in paraffin, cut into 5 mm sections and stained with hematoxylin and eosin (H&E) and periodic acid Schiff base (PAS) method. The slides were examined by light microscopy under 400× magnification for microscopic alterations of pathological significance.

### Results

The composition was evaluated based on its potential in improving renal insufficiency, decreasing renal hypertension, reducing diabetic complications, decreasing inflammation and reducing the circulating levels of kidney markers in blood

### Renal Insufficiency

Renal insufficiency is poor function of the kidneys that arises due to the decreased blood flow to the kidneys. This also arises when when the arteries that provide the kidneys with blood become narrowed—a condition termed as the renal artery stenosis. Some patients with renal insufficiency experience no symptoms or only mild symptoms.

In the present investigation, the potential of the composition comprising tetrahydrocurcuminoids, hexahydrocurcuminoids and octahydrocurcuminoids along with beta-glucogallin and C-glycosides of Pterocarpus marsupium was evaluated by their role in inhibiting Cystatin C and Kidney Injury Molecule 1.

The protease inhibitor Cystatin C is a non-glycosylated low molecular weight protein. The concentration of serum Cystatin C is mainly determined by glomerular filtration, which makes Cystatin C, an endogenous marker of glomerular filtration rate. A possible advantage of cystatin C with minor glomerular damage is that being a large molecule, blood levels of cystatin C rises sooner than creatinine. This molecule is better indicator of kidney damage and is being promoted for valuating kidney function (Grubb A, Cystatin C is Indispensable for Evaluation of Kidney Disease, EJIFCC. 2017 December; 28(4): 268-276). High cystatin C levels also lead to a number of complications which include chronic kidney disease, atherosclerosis, stroke, heart failure, coronary heart disease, angina, myocardial infarction, peripheral artery disease. Lower levels of this molecule also results in coronary artery actasia, aortic aneurysm etc.

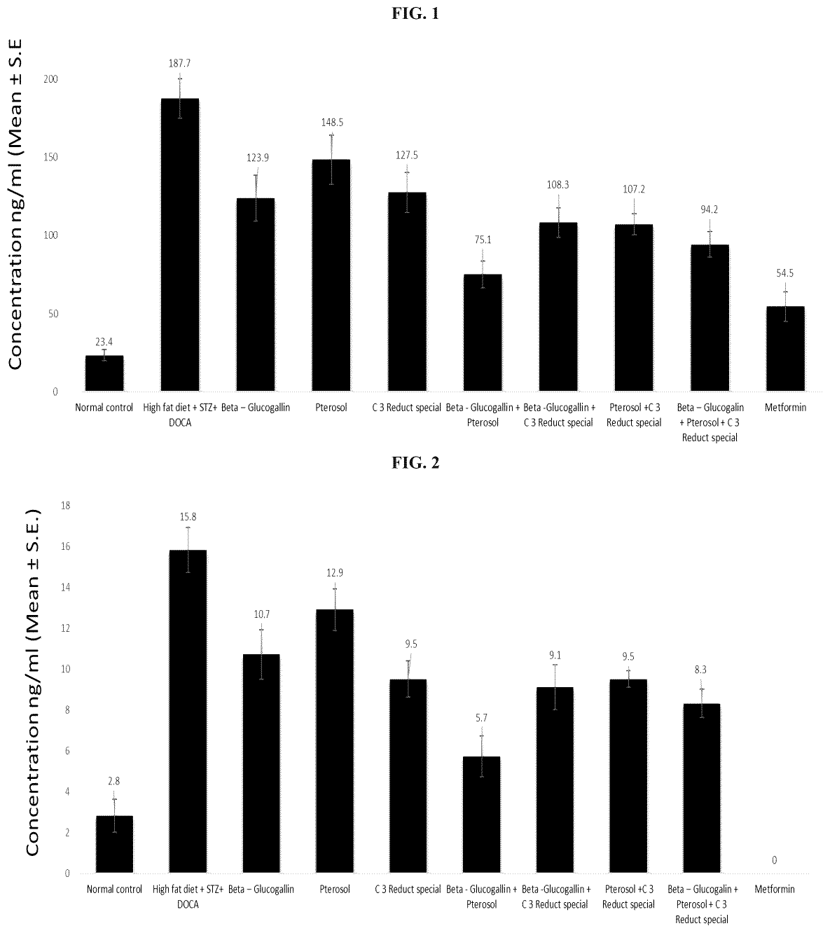

In present study, the cystatin C levels were elevated due to high fat diet, STZ and DOCA treatments. The composition comprising β-glucogallin (Saberry®) and C-glycosides from Pterocarpus marsupium (pTerosol®) showed better reduction of the elevated cystatin C levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 1)

Kidney Injury Molecule-1 is elevated in nephropathy and mediates macrophage activation (Tian et al., Kidney Injury Molecule-1 is Elevated in Nephropathy and Mediates Macrophage Activation via the Mapk Signalling Pathway, Cell Physiol Biochem 2017; 41:769-783). It is highly expressed in renal tubular cells after injury and is usually regarded as an early biomarker of acute kidney injury.

In present study, the kidney injury molecule-1 levels were elevated due to high fat diet, STZ and DOCA treatments. The composition comprising β-glucogallin (Saberry®) and C-glycosides from Pterocarpus marsupium (pTerosol®) showed better reduction of the elevated kidney injury molecule-1 levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 2)

### Renal Hypertension

Considerable evidence suggests that the intrarenal renin-angiotensin system plays an important role in nephropathy, particularly in diabetic nephropathy (Chawla et al., Role of the renin angiotensin system in diabetic nephropathy, World J Diabetes. 2010 Nov. 15; 1(5): 141-145). Angiotensin-converting enzyme (ACE) inhibitors and angiotensin II (Ang II) receptor blockers (ARBs) can attenuate progression of diabetic nephropathy in humans.

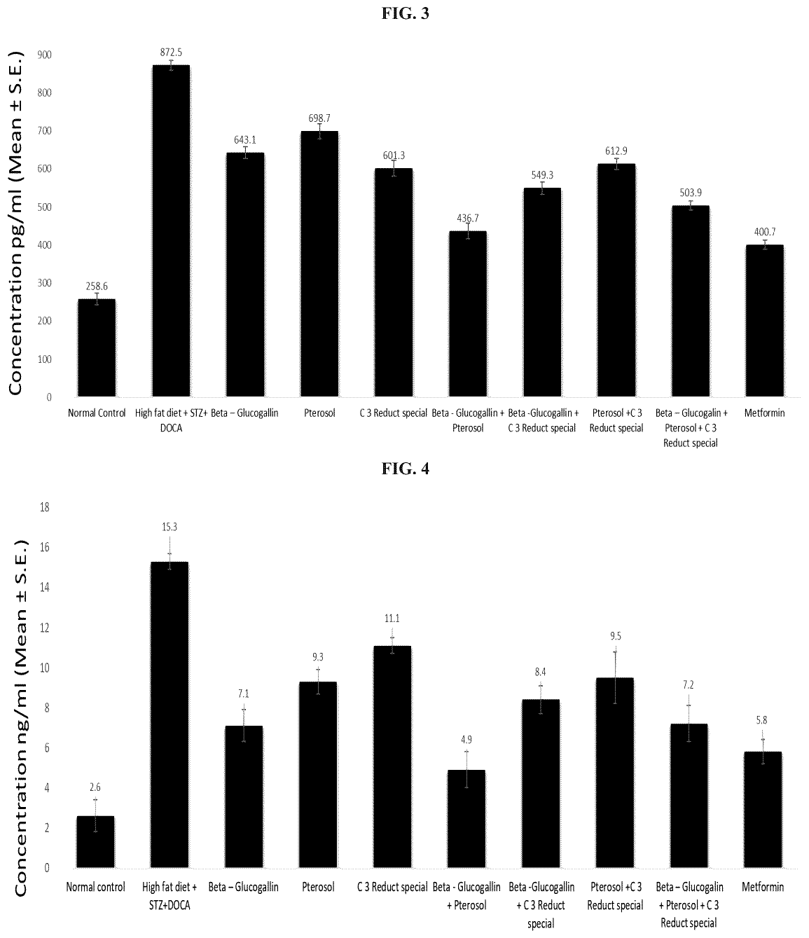

In present study, the angiotensin II levels were elevated due to high fat diet, STZ and DOCA treatments. The composition comprising β-glucogallin (Saberry®) and C-glycosides from Pterocarpus marsupium (pTerosol®) showed better reduction of the elevated angiotensin II levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 3)

### Reducing Diabetic Complication—Inflammasomes

Inflammasomes are cytosolic multiprotein oligomers of the innate immune system responsible for the activation of inflammatory responses. Nod-like receptor protein 3 (NLRP3) inflammasome and interleukin (IL) 1β activation in macrophages and dendritic cells leads to release of IL-1β, which can signal through its receptor on p cells to lead to cell death, inhibiting insulin secretory capacity. Due to the metabolic changes that occur in conditions like diabetes including insulin resistance, hyperlipidaemia and hyperglycaemia, there is a rise in damage associated molecular patterns (DAMPs) leading to an increase in inflammation, oxidative stress and NLRP3 inflammasome activation. As a result there is subsequent development of diabetic complications including diabetic nephropathy, atherosclerosis, diabetic cardiomyopathy, diabetic neuropathy and diabetic retinopathy.

In present study, the NLRP-3 levels were elevated due to high fat diet, STZ and DOCA treatments. The composition comprising β-glucogallin (Saberry®) and C-glycosides from Pterocarpus marsupium (pTerosol®) showed better reduction of the elevated NLRP-3 levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 4)

### Inflammatory Cascade

Inflammation plays a major role in the pathogenesis of nephropathy. Transforming growth factor beta (TGF-β) is an important mediator in the genesis of chronic kidney diseases (CKD), which are characterized by the accumulation of extracellular matrix (ECM) components in the glomeruli (glomerular fibrosis, glomerulosclerosis). TGF-β induces podocytopenia caused by podocyte apoptosis and detachment from the glomerular basement membrane.

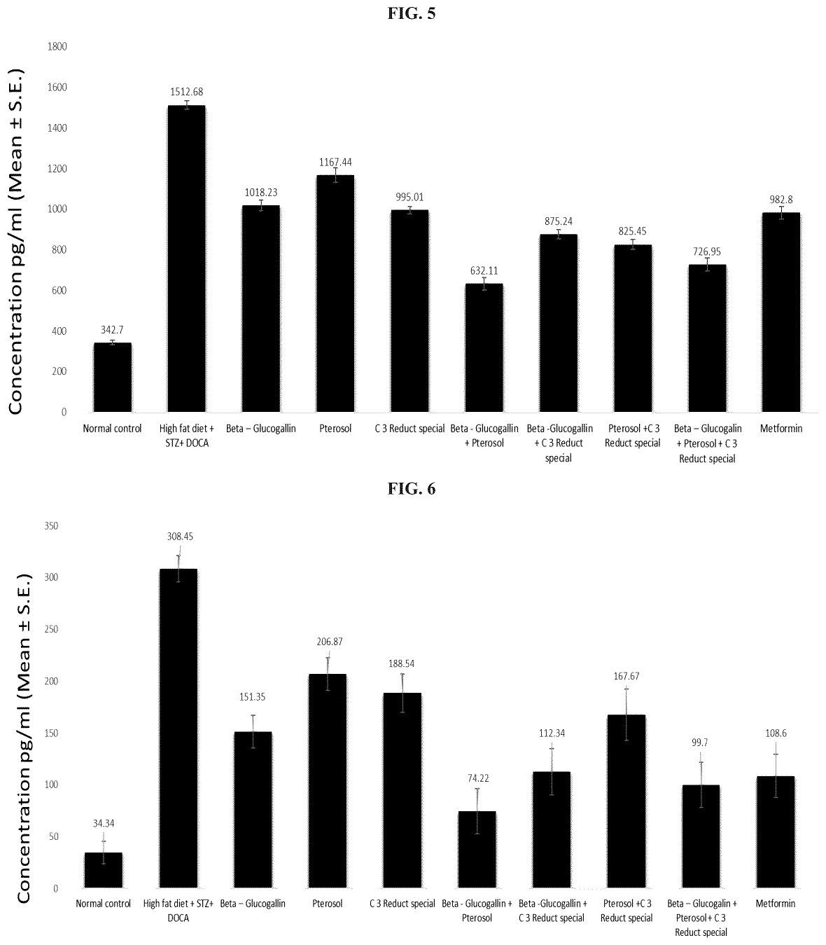

In present study, the TGF-β levels were elevated due to high fat diet, STZ and DOCA treatments. The composition comprising β-glucogallin (Saberry®) and C-glycosides from Pterocarpus marsupium (pTerosol®) showed better reduction of the elevated TGF-β levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 5)

Monocyte chemoattractant protein-1 (MCP-1/CCL2) is one of the key chemokines that regulate migration and infiltration of monocytes/macrophages. Studies in human and experimental diabetic nephropathy have shown that kidney macrophage accumulation is associated with the progression of diabetes, the development of renal injury and kidney fibrosis and the decline in renal function, suggesting that it is an inflammatory-mediated disease.

In present study, the MCP-1 levels were elevated due to high fat diet, STZ and DOCA treatments. The composition comprising β-glucogallin (Saberry®) and C-glycosides from Pterocarpus marsupium (pTerosol®) showed better reduction of the elevated MCP-1 levels followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) (FIG. 6)

### Biochemical Markers

### Creatinine

Creatinine is a waste product that comes from the normal wear and tear on muscles of the body. As the kidneys become impaired for any reason, the creatinine level in the blood will rise due to poor clearance of creatinine by the kidneys. Abnormally high levels of creatinine thus warn of possible malfunction or failure of the kidneys.

The effect of different compositions on the creatinine levels is tabulated in Table 2.

TABLE 2
Effect of compositions on creatinine levels

| | Blood Creatinine | | Urine creatinine | |
|---|---|---|---|---|
| Groups | (mg/ml) | | (mg/dl) | |
| Normal control | 1.03 ± 0.041 | | 24.67 ± 1.23 | |
| High fat diet + STZ + DOCA | 1.80 ± 0.032 | | 11.67 ± 1.06 | |
| β- Glucogallin_{+High fat diet+STZ+DOCA} | 1.53 ± 0.024 | 15%↓ | 14.33 ± 0.98 | 22.7%↑ |
| Pterosol_{+High fat diet+STZ+DOCA} | 1.45 ± 0.035 | 19.4%↓ | 15.00 ± 1.04 | 28.5%↑ |
| C3 Reduct Special_{+High fat diet+STZ+DOCA} | 1.50 ± 0.022 | 16.6%↓ | 15.07 ± 0.87 | 29.1%↑ |
| β- Glucogallin + | 1.15 ± 0.074** | 36.1%↓ | 17.20 ± 0.95** | 47.3%↑ |
| Pterosol_{+High fat diet+STZ+DOCA} | | | | |
| β- Glucogallin + C3 Reduct | 1.42 ± 0.12 | 21.1%↓ | 16.90 ± 1.21* | 44.8%↑ |
| Special_{+High fat diet+STZ+DOCA} | | | | |
| Pterosol + C3 Reduct | 1.37 ± 0.028 | 23.8%↓ | 16.60 ± 0.78* | 42.2%↑ |
| Special_{+High fat diet+STZ+DOCA} | | | | |
| β- Glucogallin + Pterosol + C3 | 1.30 ± 0.15* | 27.7%↓ | 16.88 ± 1.23* | 44.6%↑ |
| Reduct Special_{+High fat diet+STZ+DOCA} | | | | |
| Metformin (100 mg/kg)_{+High fat dict+STZ+DOCA} | 1.57 ± 0.087 | 12.7%↓ | 14.03 ± 1.32* | 20.2%↑ |

The results indicated that blood creatinine levels were elevated due to high fat diet, STZ and DOCA treatments. The composition comprising β-glucogallin (Saberry®) and C-glycosides from Pterocarpus marsupium (pTerosol®) showed better reduction of the elevated creatinine levels (36%) followed by the composition comprising 70%-80% w/w tetrahydrocurcuminoids, 10%-20%, w/w hexahydrocurcuminoids and 5%-10%, w/w octahydrocurcuminoids along with β-glucogallin (Saberry®) and C-glycosides from Pterocarpus marsupium (pTerosol®) at 28%.

### Albumin

Albumin is a blood protein found in the blood and is filtered by the kidney. The presence of increased amounts of albumin in blood indicates kidney damage. In urine albumin-to-creatinine ratio above 30 mg/g is considered higher than normal in humans.

The effect of different compositions on the albumin levels is tabulated in Table 3.

TABLE 3
Effect of compositions on albumin levels

| | |
|---|---|
| | Albumin |
| Groups | (mg/ml) |

| | | |
|---|---|---|
| Normal control | 14.33 ± 0.20 | |
| High fat diet + STZ + DOCA | 26.00 ± 0.31 | |
| β- Glucogallin_{+High fat diet+STZ+DOCA} | 18.67 ± 0.15* | 28.1%↓ |
| Pterosol_{+High fat diet+STZ+DOCA} | 18.00 ± 0.25* | 30.7%↓ |
| C3 Reduct Special_{+High fat diet+STZ+DOCA} | 19.00 ± 0.22 | 26.92%↓ |
| β- Glucogallin + Pterosol_{+High fat diet+STZ+DOCA} | 15.05 ± 0.18** | 42.1%↓ |
| β- Glucogallin + C3 Reduct | 16.33 ± 0.19** | 37.1%↓ |
| Special_{+High fat diet+STZ+DOCA} | | |
| Pterosol + C3 Reduct Special_{+High fat diet+STZ+DOCA} | 16.00 ± 0.17** | 38.4%↓ |
| β- Glucogallin + Pterosol + C3 Reduct | 15.67 ± 0.16** | 39.7%↓ |
| Special_{+High fat diet+STZ+DOCA} | | |
| Metformin (100 mg/kg)_{+High fat diet+STZ+DOCA} | 17.00 ± 0.11 | 34.6% ↓ |

The results indicated that albumin levels were elevated due to high fat diet, STZ and DOCA treatments. The composition comprising β-glucogallin (Saberry®) and C-glycosides from Pterocarpus marsupium (pTerosol®) showed better reduction of the elevated albumin levels (42%) followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) at 39.7%.

### Urea

Urea is major nitrogenous end product of protein and amino acid catabolism, produced by liver and distributed throughout intracellular and extracellular fluid. In kidneys urea is filtered out of blood by glomerulli and is partially being reabsorbed with water. If the BUN (Blood urea Nitrogen) level is higher than 100 mg/dL it points to severe kidney damage (in human) whereas decreased BUN is observed in fluid excess.

The effect of different compositions on the BUN levels is tabulated in Table 4.

TABLE 4
Effect of compositions on BUN levels

| | Blood Urea Nitrogen | |
|---|---|---|
| Groups | (mg/ml) | |
| Normal control | 15.33 ± 1.04 | |
| High fat diet + STZ + DOCA | 27.00 ± 0.98 | |
| β- Glucogallin_{+High fat diet+STZ+DOCA} | 19.67 ± 0.86* | 27.1%↓ |
| Pterosol_{+High fat diet+STZ+DOCA} | 20.80 ± 0.76 | 22.9%↓ |
| C3 Reduct Special_{+High fat diet+STZ+DOCA} | 18.60 ± 1.02** | 31.11%↓ |
| β- Glucogallin + Pterosol_{+High fat diet+STZ+DOCA} | 16.00 ± 10.87** | 40.7% ↓ |
| β- Glucogallin + C3 Reduct | 18.03 ± 0.78 | 33.2%↓ |
| Special_{+High fat diet+STZ+DOCA} | | |
| Pterosol + C3 Reduct Special_{+High fat diet+STZ+DOCA} | 17.67 ± 0.65** | 34.5%↓ |
| β- Glucogallin + Pterosol + C3 Reduct | 16.87 ± 1.03** | 37.5%↓ |
| Special_{+High fat diet+STZ+DOCA} | | |
| Metformin (100 mg/kg)_{+High fat diet+STZ+DOCA} | 17.00 ± 0.85** | 37.0%↓ |

The results indicated that blood urea nitrogen (BUN) levels were elevated due to high fat diet, STZ and DOCA treatments. The composition comprising β-glucogallin (Saberry®) and C-glycosides from Pterocarpus marsupium (pTerosol®) showed better reduction of the elevated BUN levels (40.7%) followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) at 37.5%.

### Glucose

The effect of different compositions on the blood glucose levels is tabulated in Table 5.

TABLE 5
Effect of compositions on glucose levels

| | Blood Glucose | |
|---|---|---|
| Groups | (mg/ml) | |
| Normal control | 75.67 ± 4.67 | |
| High fat diet + STZ + DOCA | 171.45 ± 5.78 | |
| β- Glucogallin_{+High fat diet+STZ+DOCA} | 122.33 ± 3.56* | 28.6%↓ |
| Pterosol_{+High fat diet+STZ+DOCA} | 118.00 ± 4.35* | 31.1%↓ |
| C3 Reduct Special_{+High fat diet+STZ+DOCA} | 126.67 ± 3.76* | 26.1%↓ |
| β- Glucogallin + Pterosol_{+High fat diet+STZ+DOCA} | 85.00 ± 4.88** | 50.4%↓ |
| β- Glucogallin + C3 Reduct | 104.67 ± 5.20* | 38.9%↓ |
| Special_{+High fat diet+STZ+DOCA} | | |
| Pterosol + C3 Reduct Special_{+High fat diet+STZ+DOCA} | 98.33 ± 4.65** | 42.6%↓ |
| β- Glucogallin + Pterosol + C3 Reduct | 101.677 ± 4.39** | 40.6%↓ |
| Special_{+High fat diet+STZ+DOCA} | | |
| Metformin (100 mg/kg)_{+High fat diet+STZ+DOCA} | 76.33 ± 5.31** | 55.47%↓ |

Glucose levels were elevated due to high fat diet, STZ and DOCA treatments. The composition comprising β-glucogallin (Saberry®) and C-glycosides from Pterocarpus marsupium (pTerosol®) showed better reduction of the elevated glucose levels (50.4%) followed by the composition comprising 70%-80% w/w tetrahydrocurcuminoids, 10%-20% w/w hexahydrocurcuminoids and 5%-10% w/w octahydrocurcuminoids along with β-glucogallin (Saberry®) and C-glycosides from Pterocarpus marsupium (pTerosol®) at 40.6%.

TABLE 6
Effect of different compositions on lipid levels

| | | Total | | |
|---|---|---|---|---|
| | Triglycerides | Cholesterol | Free fatty | Phospholipids |
| Groups | (mg/ml) | (mg/dl) | acids (mg/ml) | (mg/ml) |
| Normal control | 82.00 ± 3.68 | 35.33 ± 4.24 | 21.10 ± 1.12 | 2.62 ± 0.032 |
| High fat diet + STZ + DOCA | 148.33 ± 4.65 | 57.67 ± 4.55 | 48.34 ± 2.33 | 9.68 ± 0.024 |
| β- Glucogallin_{+High fat diet+STZ+DOCA} | 134.00 ± 4.29 | 45.00 ± 3.87 | 42.00 ± 2.10 | 6.64 ± 0.021 |
| | 9.6%↓ | 21.9%↓ | 13.1%↓ | 31.4%↓ |
| Pterosol_{+High fat diet+STZ+DOCA} | 124.33 ± 4.30 | 41.33 ± 3.76 | 36.33 ± 1.54 | 6.01 ± 0.035 |
| | 16.1%↓ | 28.3%↓ | 24.8%↓ | 37.9%↓ |
| C3 Reduct Special_{+High fat diet+STZ+DOCA} | 125.6 ± 4.29 | 42.00 ± 3.86 | 35.47 ± 1.56* | 5.00 ± 0.036* |
| | 15.2%↓ | 27.1%↓ | 26.6%↓ | 48.3%↓ |
| β- Glucogallin + Pterosol + | 120.67 ± 2.67 | 39.67 ± 3.69 | 30.50 ± 2.02* | 4.80 ± 0.045* |
| High fat diet + STZ + DOCA | 18.6%↓ | 31.2%↓ | 36.9%↓ | 50.4%↓ |
| β- Glucogallin + C3 Reduct | 122.33 ± 3.75 | 40.67 ± 3.23 | 28.33 ± 1.05** | 4.10 ± 0.041* |
| Special_{+High fat diet+STZ+DOCA} | 17.5%↓ | 29.4%↓ | 41.3%↓ | 57.6%↓ |
| Pterosol + C3 Reduct | 112.67 ± 3.98** | 38.33 ± 4.29** | 24.23 ± 2.06** | 4.00 ± 0.022* |
| Special_{+High fat diet+STZ+DOCA} | 24%↓ | 33.5%↓ | 49.8%↓ | 58.6%↓ |
| β- Glucogallin + Pterosol + C3 | 106.70 ± 3.26** | 36.45 ± 3.07* | 22.60 ± 2.07** | 3.34 ± 0.046** |
| Reduct Special_{+High fat diet+STZ+DOCA} | 28%↓ | 36.7%↓ | 53.2%↓ | 65.4%↓ |
| Metformin (100 mg/kg)_{+High fat diet+STZ+DOCA} | 122.67 ± 3.56* | 40.33 ± 3.50 | 25.34 ± 1.88** | 4.33 ± 0.023* |
| | 17.2%↓ | 30%↓ | 47.5%↓ | 55.2%↓ |

The results indicated that the compositions effectively reduced the elevated lipid levels thereby providing a complete protection against nephropathy and facilitates normal functioning of the kidney.

### Histopathology

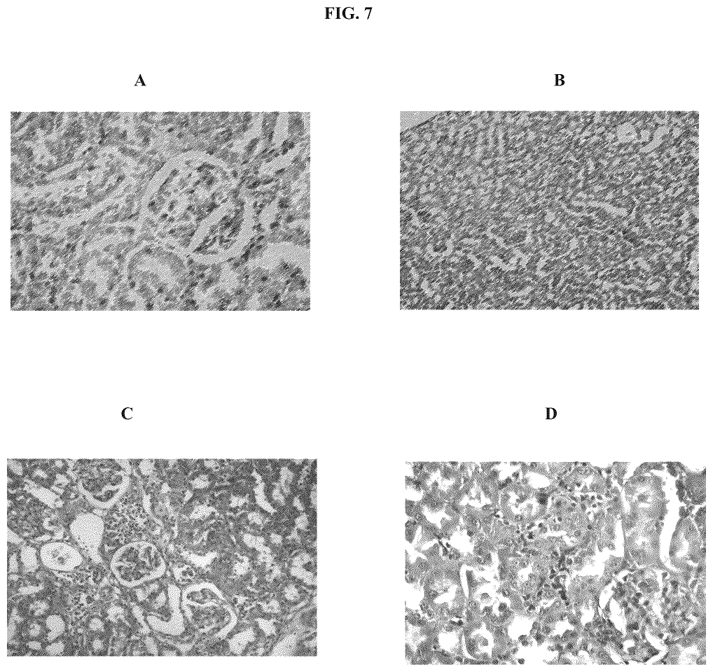

Histopathological analysis was also carried out to asses the effect of the different compositions. While the tissue section showed for the presence of normal histoarchitecture in normal controls (FIG. 7A), the tissue section showed for the presence of multifocal diffuse hydropic degeneration of kidney tubules with partial occlusion of lumen due to cell swelling. The kidney parenchyma showed widespread congestive hemorrhagic lesions in diabetic control (FIG. 7B). In mice treated with composition comprising β-glucogallin (Saberry®) and C-glycosides from Pterocarpus marsupium, the tissue section showed for the presence of mild multifocal diffuse hydropic degeneration of kidney tubules with partial occlusion of lumen due to cell swelling. Mild to moderate Multifocal renal tubular epithelial necrosis was also observed with MNC infiltration. The MNC infiltration was observed around the tubules (FIG. 7C). Further in mice treated with composition comprising tetrahydrocurcuminoids, hexahydrocurcuminoids and octahydrocurcuminoids along with β-glucogallin (Saberry®) and C-glycosides from Pterocarpus marsupium. The tissue section showed moderate cellular swelling compared to previous groups. The integrity of cells in terms of morphology was better than previous groups. Several regions of tubules also showed a regenerative response characterized by hyperplasia of renal tubular epithelium within tubules (FIG. 7D)

Overall, the composition comprising tetrahydrocurcuminoids, hexahydrocurcuminoids and octahydrocurcuminoids along with β-glucogallin and C-glycosides from Pterocarpus marsupium and the composition comprising β-glucogallin and C-glycosides from Pterocarpus marsupium were effective in improving kidney function by improving renal insufficiency, decreasing renal hypertension, reducing diabetic complications, decreasing inflammation and reducing the circulating levels of kidney markers in blood. The effective dose of the composition comprising β-glucogallin and C-glycosides from Pterocarpus marsupium in mice was about 150 mg/kg which is equivalent to the human dose of 1285.71 mg (approx. 1000 mg) and the effective dose of the composition comprising tetrahydrocurcuminoids, hexahydrocurcuminoids and octahydrocurcuminoids along with β-glucogallin and C-glycosides from Pterocarpus marsupium in mice was about 200 mg/kg which is equivalent to the human dose of 1714.29 mg (approx. 1500 mg).

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method for the therapeutic management of nephropathy in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such therapeutic management.

2. The method as in claim 1, wherein the composition further comprises at least 10% w/w 1-O-galloyl-β-D-glucose (R-glucogallin) and at least 10% w/w total mucic acid gallates and ellagic acid, isolated from Emblica officinalis.

3. The method as in claim 2, wherein the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate.

4. The method as in claim 1, wherein the composition further comprises of Pterocarpus marsupium extract standardized to contain tannins and C-glycosides containing not less than 0.5% w/w Pterocarposide, not less than 0.5% w/w Sabioside.

5. The method as in claim 1, wherein nephropathy is induced by conditions selected from the group consisting of hyperglycemia, hypercholesterolemia, elevated blood pressure and renal hypertension, drugs, autoimmune diseases, oxidative stress and inflammation.

6. The method as in claim 1, wherein the therapeutic effect is brough about by improving renal insufficiency, decreasing renal hypertension, reducing diabetic complications, decreasing inflammation and reducing the circulating levels of kidney markers in blood.

7. The method as in claim 1, where in the mammal is human.

8. A method for the maintaining normal kidney function in mammals, said method comprising step of administering a composition comprising 70%-80% w/w tetrahydrocurcuminoids, 10%-20% w/w hexahydrocurcuminoids and 5%-10% w/w octahydrocurcuminoids to mammals in need of such effect.

9. The method as in claim 8, wherein the composition further comprises at least 10% w/w 1-O-galloyl-β-D-glucose (β-glucogallin) and at least 10% w/w total mucic acid gallates and ellagic acid, isolated from Emblica officinalis.

10. The method as in claim 9, wherein the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate.

11. The method as in claim 8, wherein the composition further comprises of Pterocarpus marsupium extract standardized to contain tannis and C-glycosides containing not less than 0.5% w/w Pterocarposide, not less than 0.5% w/w Sabioside.

12. The method as in claim 8, wherein the composition maintains normal kidney function in conditions selected from the group consisting of hyperglycemia, hypercholesterolemia, elevated blood pressure and renal hypertension, drugs, autoimmune diseases, oxidative stress and inflammation.

13. The method as in claim 8, wherein the therapeutic effect is brough about by improving renal insufficiency, decreasing renal hypertension, reducing diabetic complications, decreasing inflammation and reducing the circulating levels of kidney markers in blood.

14. A method for the therapeutic management of nephropathy in mammals, said method comprising step of administering a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannins and C-glycosides containing not less than 0.5% w/w Pterocarposide, not less than 0.5% w/w Sabioside, to mammals in need of such therapeutic management.

15. The method as in claim 14, wherein the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate.

16. The method as in claim 14, wherein nephropathy is induced by conditions selected from the group consisting of hyperglycemia, hypercholesterolemia, elevated blood pressure and renal hypertension, drugs, autoimmune diseases, oxidative stress and inflammation.

17. The method as in claim 14, wherein the therapeutic effect is brough about by improving renal insufficiency, decreasing renal hypertension, reducing diabetic complications, decreasing inflammation and reducing the circulating levels of kidney markers in blood.

18. A method for the maintaining normal kidney function in mammals, said method comprising step of administering a composition comprising at least 10% w/w 1-O-galloyl-β-D-glucose (β-glucogallin) and at least 10% w/w total mucic acid gallates and ellagic acid, isolated from Emblica officinalis and Pterocarpus marsupium extract standardized to contain tannis and C-glycosides containing not less than 0.5% w/w Pterocarposide, not less than 0.5% w/w Sabioside, to mammals in need of such effect.

19. The method as in claim 18, wherein the mucic acid gallates are selected from the group consisting of mucic acid 1,4-lactone 5-O-gallate, mucic acid 2-O-gallate, mucic acid 6-methyl ester 2-O-gallate and mucic acid 1-methyl ester 2-O-gallate.

20. The method as in claim 18, wherein nephropathy is induced by conditions selected from the group consisting of hyperglycemia, hypercholesterolemia, elevated blood pressure and renal hypertension, drugs, autoimmune diseases, oxidative stress and inflammation.

21. The method as in claim 18, wherein the therapeutic effect is brough about by improving renal insufficiency, decreasing renal hypertension, reducing diabetic complications, decreasing inflammation and reducing the circulating levels of kidney markers in blood.

22. The method as in claim 18, wherein the mammal is human.
